(19) United States
(12) Patent Application Publication
(10) Pub. No.: US 2021/0290536 A1
(43) Pub. Date: Sep. 23, 2021

(54) TOPICAL EMULSION OF AN ANTICHOLINERGIC COMPOUND

(71) Applicant: Dr. August Wolff GmbH & Co. KG Arzneimittel, Bielefeld (DE)

(72) Inventors: Christoph ABELS, Bielefeld (DE); Clarissa MASUR, Bielefeld (DE)

(21) Appl. No.: 17/262,563

(22) PCT Filed: Jul. 23, 2019
(86) PCT No.: PCT/EP2019/069787

§ 371 Date: Jan. 22, 2021

(30) Foreign Application Priority Data
Jul. 24, 2018 (EP) 18185112.2

(51) Int. Cl. A61K 9/107; A61K 9/00; A61K 47/14; A61K 47/10; A61K 31/40; A61K 31/439; A61K 8/06; A61K 8/49; A61Q 15/00; A61P 17/00

(52) U.S. Cl. CPC A61K 9/107; A61K 9/0014; A61K 47/14; A61K 47/10; A61K 31/40; A61P 17/00; A61K 8/062; A61K 8/4913; A61K 8/4926; A61Q 15/00; A61K 31/439

(57) ABSTRACT

The present invention relates to a topical emulsion comprising at least one anticholinergic compound, an oil phase, a water phase and an emulsifier for use in treating patients suffering from hyperhidrosis rated as 1 or 2, preferably as 2, in the Hyperhidrosis Disease Severity Scale (HDSS). In addition the present invention relates to the non-therapeutic use of such a topical emulsion for reducing sweating in subjects who experience ordinary sweating and/or excessive sweating, wherein the subjects suffer from sweating rated as or 2, preferably as 2, in the Hyperhidrosis Disease Severity Scale (HDSS).

The present invention relates to a topical emulsion comprising at least one anticholinergic compound, an oil phase, a water phase and an emulsifier for use in treating patients suffering from hyperhidrosis rated as 1 or 2, preferably as 2, in the Hyperhidrosis Disease Severity Scale (HDSS). In addition the present invention relates to the non-therapeutic (cosmetic) use of such a topical emulsion for reducing sweating in subjects who experience ordinary sweating and/or excessive sweating, wherein the subjects suffer from sweating rated as 1 or 2, preferably as 2, in the Hyperhidrosis Disease Severity Scale (HDSS). In other words, the present invention also relates to the non-therapeutic (cosmetic) use of such a topical emulsion for reducing sweating in healthy subjects who experience excessive sweating comparable regarding the amount to patients as described above using the HDSS.

Excessive sweating or hyperhidrosis is a condition beyond what is physiologically required to maintain normal thermal regulation of the human body. Thus, hyperhidrosis is an extremely inconvenient condition negatively affecting daily life of a person suffering therefrom. Hyperhidrosis can be divided into focal and general primary hyperhidrosis. The focal is bilaterally symmetrical: hands, feet, axillae or groins. Focal hyperhidrosis from the face/head does occur but is often part of the general form. Generalized sweating usually involves both the head and trunk and in severe cases also extremities and groins/glutes. Whereas the majority of patients affected have the primary form which is hereditary, there is also a secondary form, often related to an underlying disease.

The Hyperhidrosis Disease Severity Scale (HDSS; for details see e.g. Haider A, Solish N. Focal hyperhidrosis: diagnosis and management. CMAJ 2005; 172:69-75; Solish N, Bertucci V, Dansereau A, Hong H C, Lynde C, Lupin M, et al. A comprehensive approach to the recognition, diagnosis, and severity-based treatment of focal hyperhidrosis: recommendations of the Canadian Hyperhidrosis Advisory Committee. Dermatol Surg 2007; 33(8):908-23) is a 4-point scale designed to assess the severity of hyperhidrosis in everyday clinical practice and effectiveness of hyperhidrosis treatment. It is a disease-specific diagnostic tool that provides a highly reliable qualitative measure of the severity of the patient's condition based on how it affects daily activities. A score of 3 or 4 indicates severe hyperhidrosis. A score of 1 and 2 indicates mild and moderate hyperhidrosis, respectively. An improvement of 1 HDSS score corresponds to 50% and an improvement of 2 HDSS scores to 80% reduction in sweat production.

A variety of treatments have been proposed for treating excessive sweating/hyperhidrosis which include aluminum containing antiperspirants, surgical removal of sweat glands and systemic or local treatment with anticholinergic compounds. Any of these treatments, however, entail serious disadvantages. Aluminum containing antiperspirants, in particular upon continuous use, stain or discolor the textiles coming into contact with the antiperspirant. In addition, the increasing amount of aluminum in waste water recently has also been increasing environmental concerns. On the other hand, removal of the sweat glands means surgery that may be accompanied by at least inconvenient or even serious side effects.

As an alternative to the treatments mentioned above, anticholinergic compounds such as glycopyrronium salts (also named glycopyrrolates; GP salts) have been proposed for the treatment of excessive sweating and hyperhidrosis. WO 2006/069998 discloses the use of glycopyrronium bromide together with a large variety of other active ingredients for the treatment of excessive sweating. WO 2014/134510 relates to a specific glycopyrronium tosylate salt for the treatment of excessive sweating and hyperhidrosis.

However, all of these known compositions basically suffer from at least one insufficiency selected from instability of the composition, inadequate cosmetic acceptance and skin care characteristics, systemic side effects and/or limited efficacy.

In view thereof there is still need in the prior art for new compositions being improved in treating hyperhidrosis.

Therefore, the object underlying the present invention is the provision of novel compositions for the improved treatment of excessive sweating (hyperhidrosis) and in particular of primary hyperhidrosis.

This object is achieved by the provision of the topical emulsion of the present invention comprising, next to at least one anticholinergic compound, an oil phase, a water phase, and an emulsifier, for use in treating patients suffering from hyperhidrosis rated as 1 or 2 in the Hyperhidrosis Disease Severity Scale (HDSS). In fact, it has surprisingly been found out that the topical emulsion according to the present invention is highly effective in the treatment of hyperhidrosis even in patients suffering from hyperhidrosis rated as 1 or 2, in particular as 2, in the HDSS (mild or moderate hyperhidrosis), while all prior art compositions are reported to be effective in treating only patients suffering from hyperhidrosis rated as 3 or 4 in the HDSS. Moreover, quite unexpectedly the present invention allows for an improved release of the anticholinergic compound from the emulsion, in particular as compared to gel-based formulations. This allows for an unexpectedly fast onset of action showing already statistically significant effects as early as one day after start of application, while all prior art compositions report an onset of action only after a week or even after weeks of treatment In addition, the inventive emulsion provides both for an increased stability (i.e. stable emulsion without phase separation over time) and for a cosmetic acceptance leading to a well-balanced conveniently applicable product with favorable skin care characteristics. These effects, in particular the increased stability, cannot be achieved when using glycerol as part of the emulsion. Also surprisingly, despite of the increased release of the anticholinergic compounds, the emulsion is free of side effects typical for common compositions (i.e. local and systemic side effects, such as skin irritation or even inflammation at application site, application site pain, dry mouth, dry skin, dry eye, blurred vision, mydriasis, constipation, urinary hesitation, urinary retention, and nasal dryness, known from e.g. glycopyrronium salts containing compositions). In the prior art it was not possible to sufficiently fulfill these properties all together at the same time.

According to the present invention the topical emulsion may be either a water-in-oil emulsion (also named W/O emulsion) or an oil-in-water emulsion (also named O/W emulsion). In a preferred embodiment the topical emulsion of the present invention is an oil-in-water emulsion (O/W emulsion) comprising one or more anticholinergic compound(s) as active ingredient, wherein the emulsion contains a disperse (inner) oil phase, a continuous (outer) water phase containing the anticholinergic compound, and an emulsifier. Namely, the anticholinergic compound due to its hydrophilic property and its water solubility is mainly contained in the water phase itself and in particular at the interface of the water and oil phases of the emulsion.

The oil phase of the topical emulsion of the present invention, and preferably of the O/W emulsion, may be a typical oil phase of emulsions known in the prior art. Preferred as the main (basic) oil ingredient of the oil phase, however, are linear or branched long chain fatty alcohols having 6 to 36 carbon atoms, preferably having 6 to 22 carbon atoms. More preferably according to the present invention the oil phase contains octyldodecanol because of its skin smoothening and moisturizing effects and stability against oxidation even under acidic conditions (e.g. pH 4). Octyldodecanol further improves the permeation behaviour of the emulsion of the present invention. Thus, the oil phase containing octyldodecanol is preferred because it provides improved skin care (e.g. smoothening and moisturizing) effects corresponding to an improved cosmetic acceptance of the patient or user.

The water phase comprises mainly water as solvent and the anticholinergic compound as active ingredient for treating hyperhidrosis. The water phase may also comprise water soluble/miscible compounds (e.g. lower alcohols such as ethanol or isopropanol), preservatives (such as benzyl alcohol or phenoxyethanol), pH adjusting agents or buffers (such as citric acid/citrate), as well as moisturizing agents (such as propylene glycol) and other typical hydrophilic pharmaceutical/cosmetic excipients. However, it is preferred not to incorporate glycerol itself into the emulsion of the invention because glycerol has been shown to provide a detrimental effect on stability of the emulsion.

The topical emulsions, and preferably the O/W emulsions, of the present invention contain a smaller oil phase as compared to the water phase. Preferably the oil phase ranges from 10 to 25%, and the water phase ranges from 65 to 80 wt. % of the total weight of the emulsion. Despite of the relatively small ratio of the oil phase the emulsion of the invention surprisingly provides both for better stability (no phase separation) and for improved skin care characteristics, as already mentioned above. At the same time it was unexpected that the release rate of anticholinergic compounds from the larger water phase was increased. Actually, the opposite was expected, namely a decreased release rate due to the larger water phase and the high water solubility and hydrophilic properties of the anticholinergic compounds. This allows for further reducing the amount of the anticholinergic compound leading to a better side effect profile without reducing efficacy.

The anticholinergic compounds that may be used according to the present invention are not particularly limited, and any anticholinergic compounds may be used. Preferred anticholinergic compounds are selected from the group of glycopyrronium (GP) salts, umeclidinium salts, and sofpironium salts, while GP salts are particularly preferred. GP salts chemically mean (2-cyclopentyl-2-hydroxy-2-phenylacetyl)oxy]-1,1-dimethylpyrrolidinium salts. Umeclidinium salts chemically mean salts of diphenyl-[1-(2-phenylmethoxyethyl)-1-azoniabicyclo [2.2.2]octan-4-yl]methanol. Sofpironium salts chemically mean salts of [(3R)-1-(2-ethoxy-2-oxo ethyl)-1-methylpyrro lidin-1-ium-3-yl](2R)-2-cyclopentyl-2-hydroxy-2-phenylacetate. The salts of these compounds that may be used according to the present invention are not particularly limited, and actually any salts may be used. These include the chloride, bromide, fluoride, iodide, nitrate, sulfate, sulfonate, and phosphate salts of glycopyrronium (GP), umeclidinium, and sofpironium, respectively. Also suitable are the acetate, propionate, glycolate, pyruvate, oxalate, succinate, fumarate, tartrate, citrate, benzoate, methanesulfonate, 4-methylbenzenesulfonate (tosylate), and salicylate salts of glycopyrronium (GP), umeclidinium, and sofpironium, respectively. Particularly preferably the salts are selected from the group consisting of the bromide, the acetate, and the tosylate salts of glycopyrronium (GP), umeclidinium, and sofpironium, respectively. Most preferably the salts are selected from the group consisting of the bromide, the acetate, and the tosylate salts of glycopyrronium (GP). The emulsion of the invention may contain one, two, three or more different salts of glycopyrronium (GP), umeclidinium, and sofpironium, respectively. However, preferably it contains only one salt, more preferably only one GP salt, such as the bromide, the acetate, and the tosylate salts of glycopyrronium (GP). Most preferably the anticholinergic compound is GP bromide.

The glycopyrronium (GP) salts and sofpironium salts of the present invention include the racemic mixtures thereof (umeclidinium salts are free of any chiral center). For example, the GP salt according to the present invention may be a racemic mixture of the (3R)-3-[(2S)-(2-cyclopentyl-2-hydroxy-2-phenylacetyl)oxy]-1,1-dimethylpyrrolidinium and (3S)-3-[(2R)-(2-cyclopentyl-2-hydroxy-2-phenylacetyl)oxy]-1,1-dimethylpyrro lidinium salts. Preferably a racemic mixture of (3R)-3-[(2S)-(2-cyclopentyl-2-hydroxy-2-phenylacetyl)oxy]-1,1-dimethylpyrrolidinium bromide and (3S)-3-[(2R)-(2-cyclopentyl-2-hydroxy-2-phenylacetyl)oxy]-1,1-dimethylpyrrolidinium bromide or a racemic mixture of (3R)-3-[(2S)-(2-cyclopentyl-2-hydroxy-2-phenylacetyl)oxy]-1,1-dimethylpyrro lidinium tosylate and (3S)-3-[(2R)-(2-cyclopentyl-2-hydroxy-2-phenylacetyl)oxy]-1,1-dimethylpyrrolidinium tosylate is used. The sofpironium salts used according to the present invention are those defined in WO 2014/144075 A1.

The oil-in-water emulsion according to the present invention may contain the anticholinergic compound, and in particular the GP salt such as the bromide (GPB), in an amount of 0.05 to 5 wt. %, preferably of 0.1 to 3 wt. %, more preferably of 0.2 to 2 wt. %, and even more preferably of 0.5 to 1.5 wt. %, based on the weight of the total emulsion.

When the anticholinergic compound, and in particular the GP salt such as the bromide (GPB), is contained in an amount within these ranges a high efficacy in reducing excessive sweating is achieved in patients suffering from hyperhidrosis rated as 1 or 2, preferably as 2, in the Hyperhidrosis Disease Severity Scale (HDSS), while at the same time potential undesirable side effects of GP salts can be avoided. Furthermore, surprisingly a stable emulsion can be obtained even with high amounts of GP salt (namely even with 2 to 5 wt. % and in particular with 2 to 3 wt. %). Overall, an amount of about 1.0 wt. % GP salt (in particular GPB or GP tosylate, most preferably GPB) is most preferred especially from a cost, efficacy and side effect (safety) perspective.

The emulsifier of the topical emulsion may be any emulsifier suitable to provide for a W/O or O/W emulsion. Preferably the emulsifier provides for an O/W emulsion.

A particularly preferred emulsifier according to the present invention is an emulsifier system that contains at least three different components, namely at least one macrogol glycerol fatty acid ester, at least one glycerol fatty acid ester, and at least one fatty alcohol. In the present application the term “macrogol”, as common in the pharmaceutical field, is used as a synonym for “polyethylene glycol (PEG)”. This is also supported by the skilled person's common general knowledge. Preferably, the emulsifier system comprises a combination of a macrogol glycerol fatty acid ester, a glycerol fatty acid ester, and a fatty alcohol. As the fatty alcohol it is preferred to use a mixture of two fatty alcohols. By using the specific emulsifier system in the topical (O/W) emulsion of the present invention the above mentioned effects (i.e. improved stability, increased release rate and efficacy with patients suffering from hyperhidrosis rated as 1 or 2 in the Hyperhidrosis Disease Severity Scale (HDSS), improved side effect profile and cosmetic acceptance/skin care characteristics) can be achieved.

More preferably with respect to the above described effects, the emulsifier system comprises a combination of macrogol glycerol monostearate, glycerol monostearate, and a mixture of cetyl alcohol and stearyl alcohol (e.g. with minimum 40.0 wt. % stearyl alcohol). Even more preferably, the emulsifier system comprises a combination of macrogol 20 glycerol monostearate, glycerol monostearate 40-55, and cetostearyl alcohol. Cetostearyl alcohol is a mixture of fatty acid alcohols, in particular a mixture of cetyl and stearyl alcohols. Most preferably the emulsifier system (essentially) consists of these indicated combinations.

The topical (O/W) emulsion according to the present invention may contain the respective components of the emulsifier system in the following amounts: 4.0 to 10.0 wt. %, preferably of 5.0 to 9.0 wt. %, more preferably of 6.0 to 8.0 wt. %, and even more preferably of 6.5 to 7.5 wt. % of the macrogol glycerol fatty acid ester; 1.5 to 7.5 wt. %, preferably of 2.5 to 6.5 wt. %, more preferably of 3.5 to 5.5 wt. %, and even more preferably of 4.0 to 5.0 wt. % of glycerol fatty acid ester; 5.0 to 11.0 wt. %, preferably of 6.0 to 10.0 wt. %, more preferably of 7.0 to 9.0 wt. %, and even more preferably of 7.5 to 8.5 wt. % of fatty alcohol, based on the weight of the total emulsion, respectively. By applying these preferred ranges the stability, cosmetic acceptance and efficacy is further improved.

The emulsifier system of the topical (W/O) emulsion according to the present invention may contain the respective components in the following ratios: 21 to 51 wt. %, preferably of 26 to 46 wt. %, more preferably of 31 to 41 wt. %, and even more preferably of 33 to 38 wt. % of macrogol glycerol fatty acid ester; 8 to 38 wt. %, preferably of 13 to 33 wt. %, more preferably of 18 to 28 wt. %, and even more preferably of 21 to 26 wt. % of glycerol fatty acid ester; 26 to 56 wt. %, preferably of 31 to 51 wt. %, more preferably of 36 to 46 wt. %, and even more preferably of 38 to 44 wt. % of fatty alcohol (or fatty alcohol mixture), based on the weight of the emulsifier system, respectively. By applying these preferred ratios the stability, cosmetic acceptance and efficacy is further improved.

The topical emulsion of the present invention can be used as a pharmaceutical composition/medicament or as a cosmetic composition, depending on the indication to be treated and the respective regulatory requirements.

The compositions in accordance with the present invention may contain cosmetically and pharmaceutically/dermatologically acceptable excipients known to the skilled person. These include, next to the ones already mentioned above, for example further solvents such as organic solvents, gelling agents, buffers, detergents, emulsifiers, solubilizers, humectants, fillers, bioadhesives, emollients, preservatives, bactericides, surfactants, perfumes, thickeners, softening agents, moisturizing agents, oils, fats, waxes, water, alcohols, polyols, polymers, foam stabilizers, foaming agents, anti-foaming agents, hair coating agents or other suitable components of a pharmaceutical or cosmetic preparation.

Examples for bactericides are organic acids like formic acid, sorbic acid and benzoic acid. In addition esters of p-hydroxybenzoic acid, formaldehyde-releasing agents like DMDM hydantoin, imidazolidinylurea or methyl chloroisothiazolinone, methylisothiazolinone, dibromodicyanobutane, iodopropynyl butylcarbamte, phenoxyethanol or benzyl alcohol can be used as bactericides.

Further, pharmaceutically/dermatologically acceptable excipients according to the present invention may be inorganic or organic substances for topical administration. The pH value of the formulation can be stabilized using buffer systems consisting of polyacids and their salts. Examples for such polyacids are citric acid, tartaric acid and malic acid.

In the present invention, the emulsion is preferably a dermatological composition suitable to be applied topically on the skin of a mammal. The form of the composition is not particularly limited. In preferred embodiments, the compositions are in the form of lotions, creams, sprays, shampoos, foams, or saturated pads. Preferably they can be applied using a dispenser.

Patients to be treated according to the present invention are defined as patients suffering from hyperhidrosis rated as 1 or 2, preferably as 2, in the Hyperhidrosis Disease Severity Scale (HDSS). “Hyperhidrosis” herein is intended to comprise all clinical disorders/indications associated with sweating, in particular excessive sweating. These include primary and secondary hyperhidrosis, gustatory sweating associated with Frey's syndrome, gustatory sweating associated with diabetic autonomic neuropathy, and excessive sweating in general. In particular, the following indications can be treated according to the present invention and are associated with dermatology, e.g. eccrine nevus, idiopathic unilateral focal hyperhidrosis, vascular deformities, pretibial myxedema; associated with gynaecology, e.g. postmenopausal hyperhidrosis; iatrogenic, such as associated with medicines, namely methadone or other opiates, cholinergics such as galantamine, SSRIs; associated with infection, e.g. brucellosis, HIV, chronic malaria, TBC, endocarditis; associated with surgery e.g. compensatory hyperhidrosis after sympathectomy; associated with medicines, such diabetes (hyperhidrosis due to neuropathy or hypoglycaemia), endocrine diseases (acromegaly, pheochromocytoma, hyperthyroidism, hypogonadism, insulinoma, heart failure, obesity); associated with neurology, e.g. central or peripheral lesion, Harlequin syndrome, Horner's syndrome, compensatory hyperhidrosis, Ross syndrome, Parkinson's disease, polyneuropathies; associated with oncology, such as carcinoid, lymphoma, with several malignancies; associated with orthopaedics, e.g. hyperhidrosis from amputation stump; associated with psychiatry, e.g. anxiety disorder, psychotropic drugs, social phobia.

Non-therapeutic (cosmetic) use of the topical emulsion of the present invention includes the topical application of the emulsion on the skin of a mammal in individuals experiencing inconvenient sweating rated as (or at least comparable with the state of) 1 or 2, preferably as 2, in the Hyperhidrosis Disease Severity Scale (HDSS), i.e. both including ordinary sweating and/or excessive sweating and preferably only excessive sweating.

The emulsion or composition of the present invention preferably is applied topically on the skin of a mammal. Typical application sites include the face (e.g. forehead, chin, neck and scalp), armpit, underarms, palms of the hands, soles of the feet, backs of the knees, trunk, and groin.

The topical emulsion of the present invention is topically applied onto the skin of a mammal preferably 1-4 times a day, more preferably 1-2 times a day, even more preferably 1 time per day and most preferably 2-3 times per week. It is particularly preferred to apply the topical emulsion in the above-mentioned administration regimes prior to bedtime, respectively. In a most preferred embodiment of the present invention the topical emulsion is topically applied 1 time per day, preferably prior to bedtime, within an induction period of four weeks and subsequently 2-3 times per week.

The following examples show preferred embodiments of the invention.

### EXAMPLES

The following examples have been prepared by combining the indicated ingredients using a conventional mixer (e.g. Becomix RW 320). All ingredients are commercially available. Macrogol 20 glycerol monostearate is also known as polyethylene glycol (PEG)-20 glyceryl stearate. Glycerol monostearate 40-55 is also known as glycerol monostearate 40-55% (Type II). Cetostearyl alcohol is a mixture of cetyl and stearyl alcohols.

### Preparation Example 1

| Position | Substance | g/100 g |
|---|---|---|
| 1 | Macrogol 20 glycerol monostearate | 7.00 |
| 2 | Glycerol monostearate 40-55 | 4.50 |
| 3 | Cetostearyl alcohol | 8.00 |
| 4 | Octyldodecanol | 8.00 |
| 5 | Benzyl alcohol | 1.00 |
| 6 | Glycopyrronium bromide | 1.00 |
| 7 | Citric acid, anhydrous | 0.32 |
| 8 | Sodium citrate | 0.30 |
| 9 | Propylene glycol | 3.00 |
| 10 | Purified water | 66.88 |

### Preparation Example 2

| Position | Substance | g/100 g |
|---|---|---|
| 1 | Macrogol 20 glycerol monostearate | 7.00 |
| 2 | Glycerol monostearate 40-55 | 4.50 |
| 3 | Cetostearyl alcohol | 8.00 |
| 4 | Octyldodecanol | 8.00 |
| 5 | Benzyl alcohol | 1.00 |
| 6 | Glycopyrronium bromide | 0.50 |
| 7 | Citric acid, anhydrous | 0.32 |
| 8 | Sodium citrate | 0.30 |
| 9 | Propylene glycol | 3.00 |
| 10 | Purified water | 67.38 |

### Preparation Example 3

| Position | Substance | g/100 g |
|---|---|---|
| 1 | Macrogol 20 glycerol monostearate | 7.00 |
| 2 | Glycerol monostearate 40-55 | 4.50 |
| 3 | Cetostearyl alcohol | 8.00 |
| 4 | Octyldodecanol | 8.00 |
| 5 | Benzyl alcohol | 1.00 |
| 6 | Glycopyrronium bromide | 2.00 |
| 7 | Citric acid, anhydrous | 0.32 |
| 8 | Sodium citrate | 0.30 |
| 9 | Propylene glycol | 3.00 |
| 10 | Purified water | 65.88 |

All Preparation Examples exhibited a good stability of the O/W emulsions over 36 months during stability testing (stability of emulsion in terms of phase separation and in terms of stability of active pharmaceutical ingredient (API)).

### Example—Treatment of Hyperhidrosis in Patients Rated as 2 in the Hyperhidrosis Disease Severity Scale (HDSS)

Preparation samples according to the present invention were tested in a clinical trial assessing pharmacokinetics, local and systemic tolerability and local efficacy of ascending concentrations of glycopyrronium bromide (GPB) in a topical formulation in a placebo controlled, double blind study in subjects with axillary hyperhidrosis. According to the present invention only patients suffering from hyperhidrosis rated as 2 in the Hyperhidrosis Disease Severity Scale (HDSS) were evaluated.

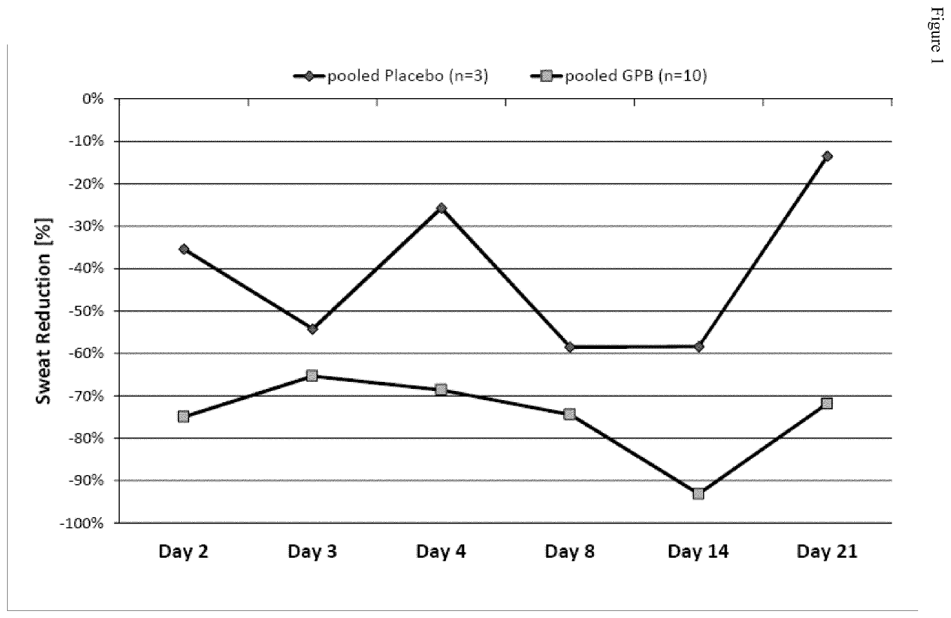

FIG. 1 shows the Percentage Sweat Reduction from baseline to Day 2-Day 21; pooled Placebo vs. pooled GPB, Cohort 1-3 (only patients starting with a HDSS of 2).

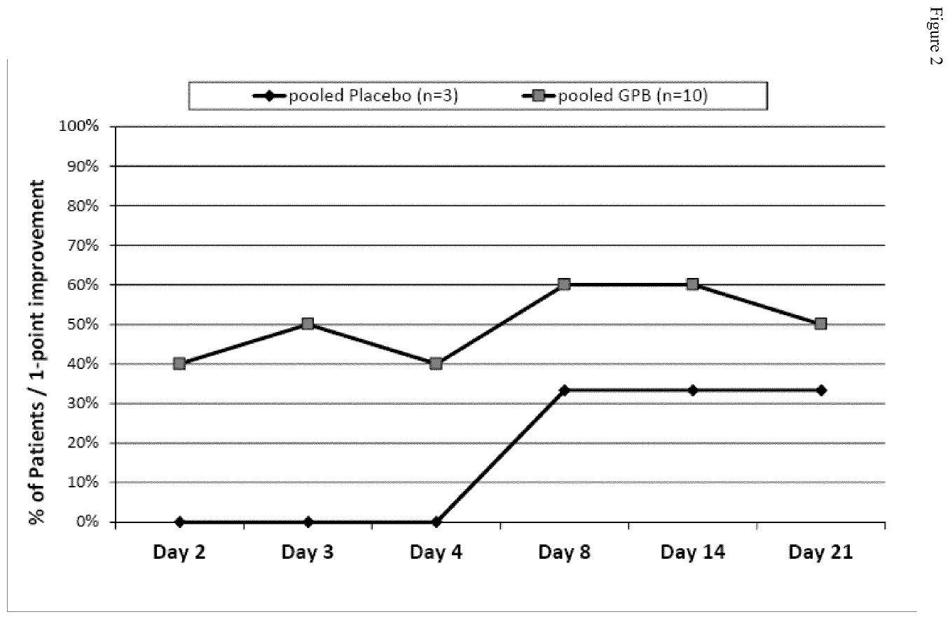

FIG. 2 shows the Percentage of Patients with 1-point improvement in HDSS from baseline to Day 2-Day 21, pooled Placebo vs. pooled GPB, Cohort 1-3 (only patients starting with a HDSS of 2).

The above Preparation Examples 1 to 3 containing 0.5 to 2.0 wt. % GPB were assessed for their efficacy in treating hyperhidrosis in patients suffering from hyperhidrosis rated as 2 in the Hyperhidrosis Disease Severity Scale (HDSS). They were topically applied once daily for 14 days on the skin of patients suffering from hyperhidrosis.

Results of Preparation Examples 1 to 3 are as follows: The sweat production after one application was reduced up to 75%, and reduction of sweat production after 13 days of treatment on day 14 was up to >90% (FIG. 1). In the four point HDSS (hyperhidrosis disease severity scale) a one-point improvement was observed for 40% of patients after one application and for 60% of patients on day 14 following 13 days of treatment (FIG. 2).

The above-mentioned results clearly show that the topical emulsion according to the present invention is highly effective in the treatment of hyperhidrosis even in patients suffering from hyperhidrosis rated as only 1 or 2, in particular as 2, in the HDSS. Thus, the topical emulsion according to the present invention is highly effective in the treatment of even mild or moderate hyperhidrosis.

Apart from the efficacy the emulsion of the present invention showed local excellent tolerability and mainly a local effect. In addition, the cosmetic acceptance of the O/W emulsion was high. The emulsion provides improved skin care characteristics including smoothening and moisturizing effects.

1. A topical emulsion comprising:
at least one anticholinergic compound, an oil phase, a water phase, and an emulsifier, for use in treating patients suffering from hyperhidrosis rated as 1 or 2 in the Hyperhidrosis Disease Severity Scale (HDSS).

2. The topical emulsion according to claim 1 in the form of an oil-in-water emulsion comprising:
a) a disperse/inner oil phase;
b) a continuous/outer water phase containing the anticholinergic compound; and
c) the emulsifier.

3. The topical emulsion according to claim 1, wherein the anticholinergic compound is selected from the group of glycopyrronium (GP) salts, umeclidinium salts, and sofpironium salts.

4. The topical emulsion according to claim 3, wherein the GP salt is a racemic mixture of (3R)-3-[(2S)-(2-cyclopentyl-2-hydroxy-2-phenylacetyl)oxy]-1,1-dimethylpyrrolidinium bromide and (3S)-3-[(2R)-(2-cyclopentyl-2-hydroxy-2-phenylacetyl)oxy]-1,1-dimethylpyrrolidinium bromide; or a racemic mixture of (3R)-3-[(2S)-(2-cyclopentyl-2-hydroxy-2-phenylacetyl)oxy]-1,1-dimethylpyrrolidinium tosylate and (3S)-3-[(2R)-(2-cyclopentyl-2-hydroxy-2-phenylacetyl)oxy]-1,1-dimethylpyrrolidinium tosylate.

5. The topical emulsion according to claim 3, wherein the GP salt is present in an amount of 0.05 to 5 wt. %, based on the weight of the total emulsion.

6. The topical emulsion according to claim 1, wherein the emulsifier is an emulsifier system comprising:
at least one macrogol glycerol fatty acid ester,
at least one glycerol fatty acid ester, and
at least one fatty alcohol.

7. The topical emulsion according to claim 6 comprising as the emulsifier system a combination of:
macrogol glycerol monostearate,
glycerol monostearate, and
a mixture of cetyl and stearyl alcohol.

8. The topical emulsion for use according to claim 6 comprising as the emulsifier system a combination of:
macrogol 20 glycerol monostearate,
glycerol monostearate 40-55, and
cetostearyl alcohol.

9. The topical emulsion according to claim 6 comprising,
an amount of 4.0 to 10.0 wt. % of the macrogol glycerol fatty acid ester,
an amount of 1.5 to 7.5 wt. % of glycerol fatty acid ester, and
an amount of 5.0 to 11.0 wt. % of fatty alcohol, based on the weight of the total emulsion, respectively.

10. The topical emulsion according to claim 6, wherein the emulsifier system comprises:
an amount of 21 to 51 wt. % of macrogol glycerol fatty acid ester,
an amount of 8 to 38 wt. % of glycerol fatty acid ester, and
an amount of 26 to 56 wt. % of fatty alcohol, based on the weight of the emulsifier system.

11. The topical emulsion according to claim 1, wherein the hyperhidrosis to be treated is selected from the group consisting of primary and secondary hyperhidrosis, gustatory sweating associated with Frey's syndrome, gustatory sweating associated with diabetic autonomic neuropathy, and excessive sweating.

12. The topical emulsion according to claim 1, wherein the emulsion is topically applied onto the skin of a mammal 1-4 times a day.

13. The topical emulsion according to claim 12, wherein the emulsion is topically applied 1 time per day within an induction period of four weeks and subsequently 2-3 times per week.

14. A pharmaceutical composition comprising the topical emulsion according to claim 1 and one or more pharmaceutically acceptable excipients.

15. A non-therapeutic use of a topical emulsion comprising:
at least one anticholinergic compound, an oil phase, a water phase, and an emulsifier, for reducing sweating in subjects who experience ordinary sweating and/or excessive sweating, wherein the subjects suffer from sweating rated as 1 or 2 in the Hyperhidrosis Disease Severity Scale (HDSS).

16. The topical emulsion according to claim 3, wherein the anticholinergic compound is selected from the group consisting of the bromide, the acetate, and the tosylate salts of glycopyrronium (GP), umeclidinium, and sofpironium, respectively.

17. The topical emulsion according to claim 5, wherein the GP salt is present in an amount of 0.5 to 1.5 wt. %, based on the weight of the total emulsion.

18. The topical emulsion according to claim 6 comprising:
an amount of 6.5 to 7.5 wt. % of the macrogol glycerol fatty acid ester,
an amount of 4.0 to 5.0 wt. % of glycerol fatty acid ester, and
an amount of 7.5 to 8.5 wt. % of fatty alcohol, based on the weight of the total emulsion, respectively.

19. The topical emulsion according to claim 6, wherein the emulsifier system comprises:
an amount of 33 to 38 wt. % of macrogol glycerol fatty acid ester,
an amount of 21 to 26 wt. % of glycerol fatty acid ester, and
an amount of 38 to 44 wt. % of fatty alcohol, based on the weight of the emulsifier system

20. The topical emulsion according to claim 1, wherein the emulsion is topically applied onto the skin of a mammal 2-3 times per week.
